(19) United States
(12) Patent Application Publication
(10) Pub. No.: US 2005/0215892 A1
(43) Pub. Date: Sep. 29, 2005

(54) System and method for transducer array cooling through forced convection

(75) Inventors: Charles D. Emery, Renton, WA (US); Zoran B. Banjanin, Newcastle, WA (US); Chi-Yin Lee, Bellevue, WA (US); Alampallam R. Ramachandran, Sammamish, WA (US); John R. Dennis, Renton, WA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/805,783

(22) Filed: Mar. 22, 2004

(51) Int. Cl. A61B 8/00; A61B 8/06

(52) U.S. Cl. 600/437; 600/459

(57) ABSTRACT

A system and method for cooling an entirely or partially immersed mechanical or other type of transducer array is disclosed. Motion/flow of the immersion fluid is induced either by motion of the mechanical transducer itself, where the transducer is of the mechanically movable type, or by a separate motion-inducing mechanism located in or coupled with the fluid-filled, or partially filled, array housing. The resultant fluid flow/motion increases, i.e. more efficiently utilizes, the thermal carrying capacity of the immersion fluid by more uniformly distributing the thermal energy convected from the transducer array throughout the fluid volume. This results in an improved ability to cool the transducer array. The disclosed cooling system and method may be used in such a way so as to not substantially inhibit operation of the transducer array.

### BACKGROUND

Medical ultrasound imaging has become a popular means for visualizing and medically diagnosing the condition and health of interior regions of the human body. With this technique an acoustic transducer probe, which is attached to an ultrasound system console via an interconnection cable or wireless connection, is held against the patient's tissue by the sonographer whereupon it emits and receives focused ultrasound waves in a scanning fashion. The scanned ultrasound waves, or ultrasound beams, allow the systematic creation of image slices of the patients internal tissues for display on the ultrasound console. The technique is quick, painless, fairly inexpensive and safe, even for such uses as fetal imaging.

In order to get the best performance from an ultrasound system and its associated transducers it is desirable that the transducers used to emit and receive ultrasonic pulses be capable of operating at the maximum acoustic intensity allowable by the U.S. Food and Drug Administration (FDA). This will help maximize the signal to noise ratio for the given system and transducer, help achieve the best possible acoustic penetration, and ensure that imaging performance is not limited by the inability to emit the full allowable acoustic intensity. Further, this will allow for maximum performance of the various imaging modes such as color flow, Natural Tissue Harmonic Imaging (“NTHI”) and spectral Doppler. In NTHI mode, the transducer is excited at one frequency and receives the acoustic echoes at a second frequency, typically the second harmonic, in order to account for the non-linear propagation of acoustic waves through tissue and the harmonics created thereby. At the same time, there are practical and regulatory limits on the allowable surface temperature that the transducer may attain as it performs its imaging functions. For example, the Underwriters Laboratory (U.L.) Standard #UL544 “Standard for Safety: Medical and Dental Equipment” specifies an upper limit of 41° C. for the transducer portion contacting the patient's skin, while the International Electrotechnical Commission (“IEC”) specification IEC 60601-2-37 specifies an upper limit of 43° C. In addition, sonographers prefer to grip a transducer case which is comfortably cool, thereby preventing excess perspiration in their hands and a potential to lose their grip on the device. Further, increased internal temperatures may affect the operational characteristics or capabilities of the transducer components, reducing their efficiency and/or operating capabilities. For example, CMOS integrated circuits, which may be utilized as part of the control circuitry in the transducer, operate faster and more efficiently at lower temperatures.

Additionally, the introduction of Coded Excitation Transmitting, such as “Chirp transmit waveforms”, Multi-focus (dynamic transmit focus) and high frame rate imaging modes has significantly increased the requirements for transmit power of the transducer. This increase in operating power has necessarily led to an increase in operating temperatures.

Given that it is desirable to be able to operate at the maximum allowable acoustic intensity and also desirable to control the internal transducer operating temperatures as well as the surface temperature distribution of the patient and user-contacting portions of the transducer's surfaces, thermal engineering is a serious consideration during transducer design.

### BRIEF DESCRIPTION OF THE DRAWINGS

FIG. 1 depicts block diagram of an exemplary transducer according to one embodiment.

FIG. 2 depicts a block diagram of an exemplary transducer module according to one embodiment for use with the transducer of FIG. 1.

FIG. 3 depicts a flow chart showing exemplary operation of the transducer of FIG. 1.

### DETAILED DESCRIPTION OF THE DRAWINGS AND PRESENTLY PREFERRED EMBODIMENTS

A system and method for cooling an immersed or partially immersed mechanical transducer array is disclosed. Motion/flow of the immersion fluid is induced either by motion of the mechanical transducer itself or by a separate motion-inducing mechanism located in or coupled with the fluid-filled array housing. Herein, the phrase “coupled with” is defined to mean directly connected to or indirectly connected through one or more intermediate components. Such intermediate components may include both hardware and software based components. Further, the term fluid, as used herein, includes both gasses, liquids, including liquids which undergo phase transition to a gas form, and solids which undergo phase transition to liquid or gas form, or mixtures or intermediate transitional forms thereof. The resultant fluid flow/motion increases, i.e. more efficiently utilizes, the thermal carrying capacity of the immersion fluid by more uniformly distributing the thermal energy convected from the transducer array throughout the fluid volume. This results in an improved ability to cool the transducer array. The heat that is convected from the transducer array may then be removed from the fluid via other techniques, as described below. The disclosed cooling system and method may be used in such a way so as to not substantially inhibit operation of the transducer array. The disclosed embodiments are especially applicable to wireless transducer implementations which do not feature an interconnect cable which can be used, in addition to electrical/signal connections, to connect internal cooling devices of the transducer with external heat exchangers.

Mechanical transducer arrays, also known as wobblers, are arrays, piezoelectric, micromechanical or otherwise implemented, that may be mechanically actuated so as to allow movement of the scanning area without requiring movement of the overall transducer by the operator. Such arrays are typically contained within a fluid filled housing. The fluid operates to acoustically couple the transducer array with the patient such that the emitted ultrasonic energy and received ultrasonic echoes pass between the array and the patient substantially unimpeded, as will be described in more detail below. Although the disclosed embodiments relate to mechanical transducers, a.k.a. wobblers, it will be appreciated that electronically manipulatable, i.e. steering or focus, transducers, such as 1.5D, 1.75D or 2D arrays, whether piezoelectric, micro-mechanical or otherwise, either fixed or mechanically movable as described above, may also be cooled by similarly immersing, either entirely or partially, the transducer array and using the disclosed convection cooling techniques. For information regarding alternate methods of removing heat from non-mechanical transducer arrays, see U.S. Pat. application Ser. No. 10/183,302, entitled “SYSTEM AND METHOD FOR IMPROVED TRANSDUCER THERMAL DESIGN USING THERMO-ELECTRIC COOLING” filed Jun. 27, 2002, now U.S. Pat. No. ______, herein incorporated by reference, and U.S. Pat. No. 5,560,362, entitled “ACTIVE THERMAL CONTROL OF ULTRASOUND TRANSDUCERS”. For more information regarding removing beat from the transducer electronics, see U.S. Pat. Application Ser. No. ______, entitled “SYSTEM AND METHOD FOR ACTIVELY COOLING TRANSDUCER ASSEMBLY ELECTRONICS” (Attorney Ref. No. 2003P19702US), filed Mar. 15, 2004, herein incorporated by reference. The methods disclosed in these references may be used to cool the immersion fluid.

Mechanical transducers have been developed for special purposes such as generating 3D or 4D ultrasound images. There are several types of these transducers depending upon what type of mechanical movement is implemented, such as rocking, also referred to as Mechanically Rocking Arrays (“MRA”), and linear moving, also referred to as Mechanically Linearly Moving Arrays (“MLA”), etc. For example, in operation, the sonographer holds the transducer steady relative to the patient while the transducer array is mechanically moved to sweep the elevation of the volume being examined for the purpose of creating a 3D or 4D image.

The disclosed embodiments take advantage of the transducer array being immersed, entirely or partially, in a fluid for the additional purpose of cooling the transducer. Liquid immersion cooling is known in microelectronics industry as a means to cool high power integrated circuits. See “Direct liquid immersion cooling for high power density microelectronics”, Robert E. Simmons, Electronic Cooling Applications, available at http://www.electronics-cooling.com/Resources/EC_Articles/MAY96/may96_{—}04.htm (last accessed on Jan. 28, 2004). Such cooling systems circulate a coolant around the electronics to be cooled, thereby carrying away the generated thermal energy to some form of external heat exchanger. While this provides effective cooling, such systems also require complex plumbing and heat exchange mechanisms.

The disclosed embodiments utilize the immersion fluid contained within the transducer array housing. While some measure of heat transfer occurs just by having the transducer array immersed, entirely or partially, in a fluid, the thermal energy generated by the transducer array tends to be localized within the fluid volume in proximity to the transducer array, i.e. in “hot spots.” Improved cooling may be achieved by inducing movement in the fluid to more uniformly redistribute the thermal energy that is generated by the array throughout the fluid volume of the housing. It will be appreciated that the fluid used to immerse the transducer may be of a type selected for both its acoustic and thermal characteristics. For example, the fluid may also be used as a matching layer to match acoustic impedance. Further, where the fluid is or includes a solid which undergoes a phase change to either a liquid or gas phase, it will be appreciated that movement may be induced in the resultant liquid or gas once the solid has undergone the change in phase. As will be described below, the phase transition itself may actually induce such movement.

Often during operation of a mechanical transducer array, the sonographer will freeze the operation of the transducer array for the purpose of studying, saving or printing the acquired images. During these periods of inactivity, the operation of the transducer array ceases and the array begins to cool down. In one embodiment, the cooling of the transducer array is improved by utilizing the mechanical movement capability of transducer array. During freeze periods while the transducer array is not emitting ultrasonic energy, the movement mechanism of the transducer array is actuated so as to move the array within the fluid. The degree of movement may be preset, such as moving the array back and forth throughout its complete range of motion or a more limited movement may be used depending upon the implementation and the desired thermal effect. Further the speed of the movement may be fast or slow. In one embodiment, the motion and speed are dynamically controlled based on the desired thermal effect. Movement of the array induces fluid movement across the surface of the array and causes improved convection of thermal energy from the array to the fluid. Further, the movement of array induces movement of the fluid and acts to mix or stir the fluid, thereby causing any localized thermal energy, i.e. “hot spots” to be redistributed throughout the fluid volume, resulting in an overall increased thermal carrying capacity. As discussed above, other techniques may then be used to remove the thermal energy from the fluid.

In one embodiment, the movement of the transducer array during freeze periods is limited such that once the thermal energy is uniformly distributed between the transducer array and the fluid, the speed or degree of the array movement is altered, for example slowed or stopped, and/or the range of movement is altered, for example the range is narrowed. This prevents the actuating mechanism itself from contributing excess additional thermal energy to the fluid and thereby reducing its ability to carry thermal energy away from the transducer array.

As described, other additional mechanisms may further be provided to remove thermal energy from the fluid and thereby enhance the fluid's ability to remove thermal energy from the transducer array.

In one embodiment, the actuating of the transducer array during freeze periods for the purposes of cooling may begin as soon as the operator initiates the freeze. Under normal operating conditions, actuation of the transducer array for the purposes of imaging may require an initialization procedure, such as a procedure to home the initial position of the transducer array or otherwise initialize the imaging and driving software. For example, 3D or 4D imaging operations may require homing the position of the transducer to a known origin so as to be able to properly process the data generated by the transducer. These initialization procedures typically require a significant amount of time to complete but may be unnecessary for the purposes of transducer cooling. In the current embodiment, when the operator freezes operation, the system is able to bypass any initialization procedures that may be required for imaging purposes and initiate transducer array movement for cooling purposes substantially immediately. In an alternate embodiment, the initialization procedures may be deferred until after sufficient cooling has occurred. Once sufficient cooling has occurred, the initialization procedures may be performed to prepare the transducer for the next imaging operation.

In yet another embodiment, transducer array temperature may be monitored and the system may force a freeze period if the transducer temperature exceeds a predetermined threshold. During this forced freeze period, the transducer array is actuated as described above to cool the array down to a temperature within prescribed operating limits. For example, some ultrasound systems permit a High Transmit Power mode wherein the transmission power of the transducer array is allowed to exceed normal operating limits for short periods of time for improved deep imaging. An exemplary High Transmit Power mode is further described in U.S. patent application Ser. No. 10/304,350, entitled “IMPROVING DIAGNOSTIC ULTRASOUND THROUGH HIGH TRANSMIT POWER INTERLEAVED WITH LOW TRANSMIT POWER”, filed on Nov. 26, 2002, herein incorporated by reference. Such increases in transmission power, however, lead to significant increases in the temperature of the transducer array and the overall temperature of the transducer, including those portions which contact the patient. In such systems which offer this High Transmit Power mode, the short operating period during which the High Transmit Power mode is active may be followed by the forced freeze and cooling method described above to return the transducer array temperature back to within prescribed limits.

In an alternate embodiment, the transducer operating temperature is monitored and a freeze period is forced when the monitored temperature deviates from defined temperature threshold. This temperature threshold may be a statically defined value or may be dynamic, for example, based on the particular operating mode of the transducer. In another embodiment, a timer may be provided which limits the amount of time that the transducer may be used before a freeze period is forced. The timer limit may be a fixed value or may be dynamically determined based on, for example, the mode of operation of the transducer. Further, a combination of the operator-activated freeze periods and forced freeze periods may be implemented.

In another embodiment, a separate mechanism is provided to cause fluid movement within the housing and achieve the requisite uniform thermal redistribution. It will be appreciated that these separate mechanisms described below may be used alone, such as with fixed/non-moving transducer arrays, or in combination with actuating the transducer array as described above for the purposes of cooling. In embodiments using a separate cooling mechanism as described below, it may not be necessary to cease normal operation of the transducer when the fluid movement mechanism is operating. In these embodiments, the fluid movement mechanism may be designed so as not to interfere with the operation of the transducer. Further, in these embodiments, the fluid movement mechanism may be activated, as described above, during idle periods, based on sensed temperature or based on a timer, or a combination thereof, or they may continuously operate. The mechanism for inducing fluid movement may be either active or passive. In embodiments which utilize a phase-changing solid, alone or in combination with other gasses or liquids, the fluid movement mechanism may further include a mechanism to cause the requisite change in phase prior to inducing fluid movement.

Active mechanisms include motorized pumps, paddles or rotors which re-circulate the fluid contained within the housing. It will be appreciated that any mechanism that can induce fluid movement may be used, including “electrokinetic” based pumps which may use micromechanical devices to move the fluid. The mechanism, including the fluid moving device (paddle, stirrer, etc) as well as the motor or other actuating mechanism, may all be contained within transducer array housing. Alternatively, the actuating mechanism may be outside the transducer array housing and mechanically or otherwise coupled with the fluid moving device contained within the housing, such as via a direct mechanical connection or via a magnetic or other connection.

Passive mechanisms for inducing fluid flow utilize the natural movement of the transducer or the thermal energy in the fluid itself to induce movement. In one embodiment, the thermal differential between the localized “hot spots” and the rest of the fluid volume is utilized to induce movement in the fluid and thereby redistribute the thermal energy. This may be achieved by using an immersion fluid of which the density changes with temperature or use of a fluid, alone or in combination with other liquids, solids or gasses, which undergoes a change in phase, such as from liquid to gas, solid to liquid or solid to gas. The shape of the transducer array housing may also be designed to promote fluid movement, such a by the inclusion of internal fins or other features. In another embodiment, the movement of the transducer by the sonographer is utilized to induce fluid movement, such as by the inclusion of a gravitationally or inertially-responsive mixing or stirring device in the transducer array housing which actuates when the orientation of the transducer is changed by the sonographer or the transducer is otherwise moved.

In all of the disclosed embodiments, the disclosed mechanisms operate at a rate which achieves the desired redistribution without adding unnecessary energy and without interfering with normal imaging operation of the transducer array. For example, the movement of the fluid may need to be stopped or damped during imaging operations. In alternative embodiments, where the addition of excess energy is not a concern, the disclosed mechanisms may operate at a rate commensurate with the desired thermal effect.

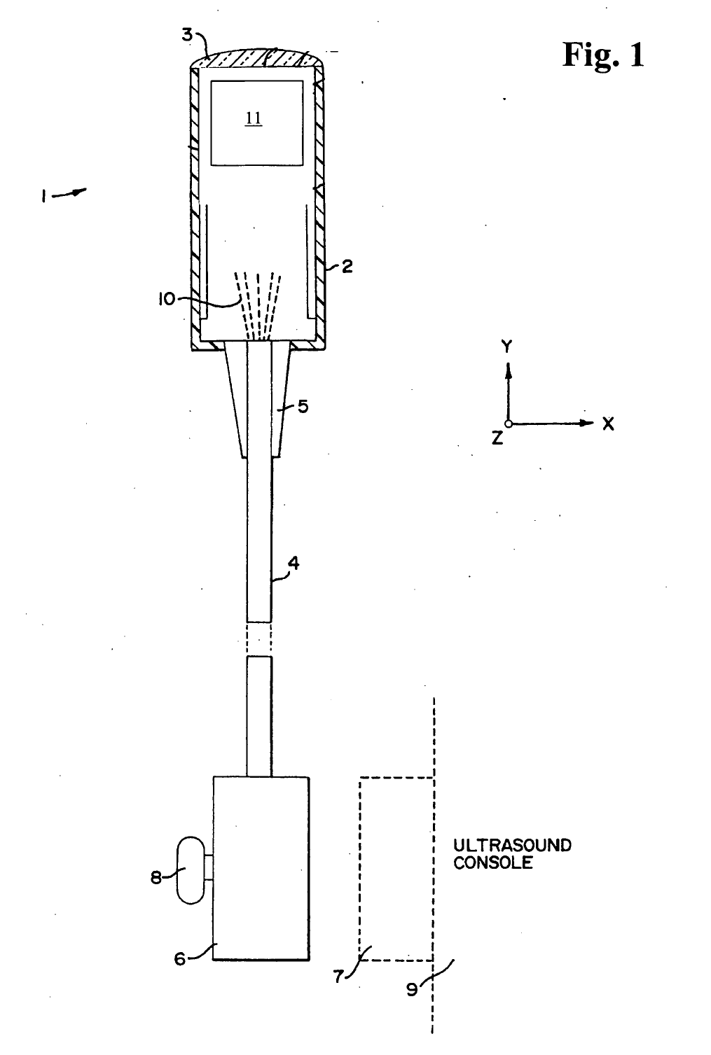

FIG. 1 shows an exemplary medical ultrasound transducer 1 in schematic sectional view. Transducer 1 has a typically polymeric external case 2 which is gripped by the sonographer. The top of the transducer (+Y end) can be seen to have the typical acoustic lens 3 which serves to focus the ultrasound beam in the X-Y plane as it passes into the subject patient. Focusing in the Y-Z plane is done via electronic phase delays between the various piezoelements which are arranged on a Z-axis pitch and spacing passing into and out of the paper as is usual for phased array transducers. The bottom or back of the transducer 1 has emanating from it a flexible coaxial cable bundle 4. The cable 4 is shown in broken view at its midpoint to indicate its considerable length, usually on the order of 6 to 12 feet. Where cable 4 exits from the transducer 1, and specifically where it exits from the transducer case 2, can be seen a flexible strain relief 5. Strain reliefs are usually fabricated from a flexible rubber, such as silicone rubber, and they serve to prevent damage to the cable 4 or chemical leakage into the case 2 at the point of cable/case juncture particularly as cable 4 is flexed by the user.

A transducer cable connector 6 can be seen at the termination of the cable 4 (−Y end). The connector 6 is usually of a mass-actuated design and has an appropriate rotatable actuation knob 8 for that function. To the right of the transducer's connector 6 are shown in phantom a mating ultrasound system connector 7 mounted on an ultrasound system console 9. To use the transducer the sonographer would plug connector 6 into mating connector 7 (connectors shown unmated) thereby electrically connecting the transducer 1 to the ultrasound system console 9.

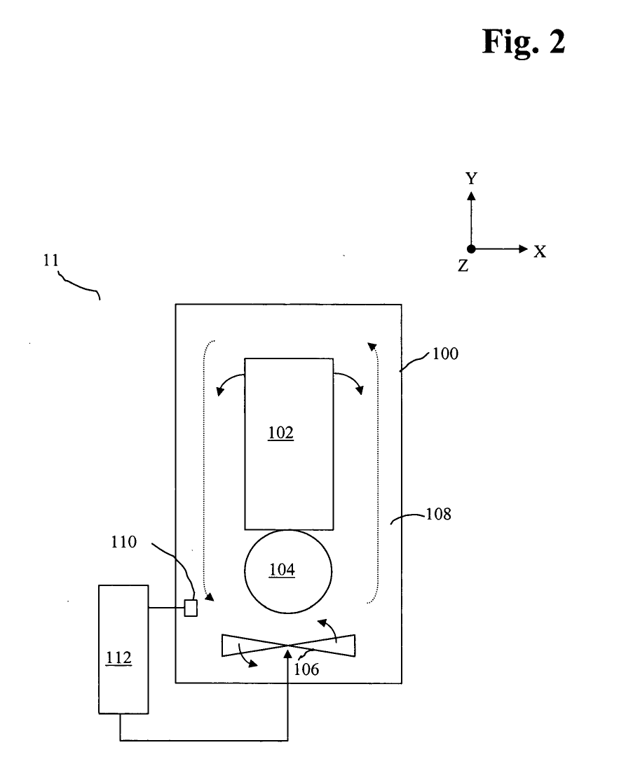

In the interior portion of the bottom of transducer 1, inside of polymeric case 2, portions of numerous electrical interconnects 10 (indicated by partial dotted lines) run from the transducer device 1 into the cable 4 and, in turn, into the connector 6. Generally a large number of interconnects 10 comprising coaxial wires of controlled impedance are provided in cable 4 to carry the electrical impulses transmitted to and received from the individual piezoelements making up the phased array. The details of how the interconnects 10 are mated to the piezoelements or to the connector are not shown as it is not critical to the understanding of this invention. It should be generally understood that numerous interconnects 10 pass from the transducer 1 and its piezoelements through the cable to the connector 6 and these serve an electrical function. Interconnects 10 must physically be routed through the interior of the back of the transducer case 2, and around whatever other means, thermal or otherwise, are located therein. The transducer module 11, containing the entirely or partially immersed piezoelectric transducer array (shown in more detail in FIG. 2), is packaged and operated inside the confines of the polymeric case 2. It will be appreciated that, as was described above, the disclosed embodiments may be used with piezoelectric, micromechanical or other types of transducers. Further, the disclosed embodiments may be used with transducers which are connected to the ultrasound system via a wired or wireless connection.

FIG. 2 shows the transducer module 11 in mode detail. The transducer module 11 includes a housing 100. The housing 100 is sealed to contain the immersion fluid 108, described in more detail below, while allowing for electrical interconnections between the components located in the housing and those components of the transducer located outside of the housing 100. Further, the housing 100 includes at least one portion that acts as an acoustic window allowing acoustic emissions from the piezoelectric or micromechanical transducer array 102 to exit the housing and return echoes to enter the housing substantially unimpeded.

Within the housing 100 is located the piezo-electric transducer array 102, the actuating mechanism 104 wherein the transducer array 102 is of the movable type described above, and the immersion fluid 108. It will be appreciated that the actuating mechanism 104 may also be located outside of the housing 100 and mechanically or otherwise coupled with the movable transducer array 102. In one embodiment, the housing 100 also contains a fluid movement inducing mechanism 106. In this embodiment, the drive mechanism (not shown), if required, for operating the fluid movement inducing mechanism 106 may be located within the housing 100 or external to the housing 100 and mechanically or otherwise coupled with the fluid movement inducing mechanism 106.

The piezo-electric transducer array 102 is of a design known in the art. The array 102 is immersed, either entirely or partially, in the fluid 108 and sheds operating heat into the surrounding fluid 108 as described above. As was described, during freeze periods, the transducer array 102 actuating mechanism 104 may be activated to induce movement in the fluid 108 thereby more uniformly distributing the thermal energy shed by the array 102. Alternatively, or in addition to operating the actuating mechanism 104, a fluid movement inducing mechanism 106, either active or passive as described above, may be used to induce movement in fluid 108 to achieve the same result or enhance the redistribution of the thermal energy.

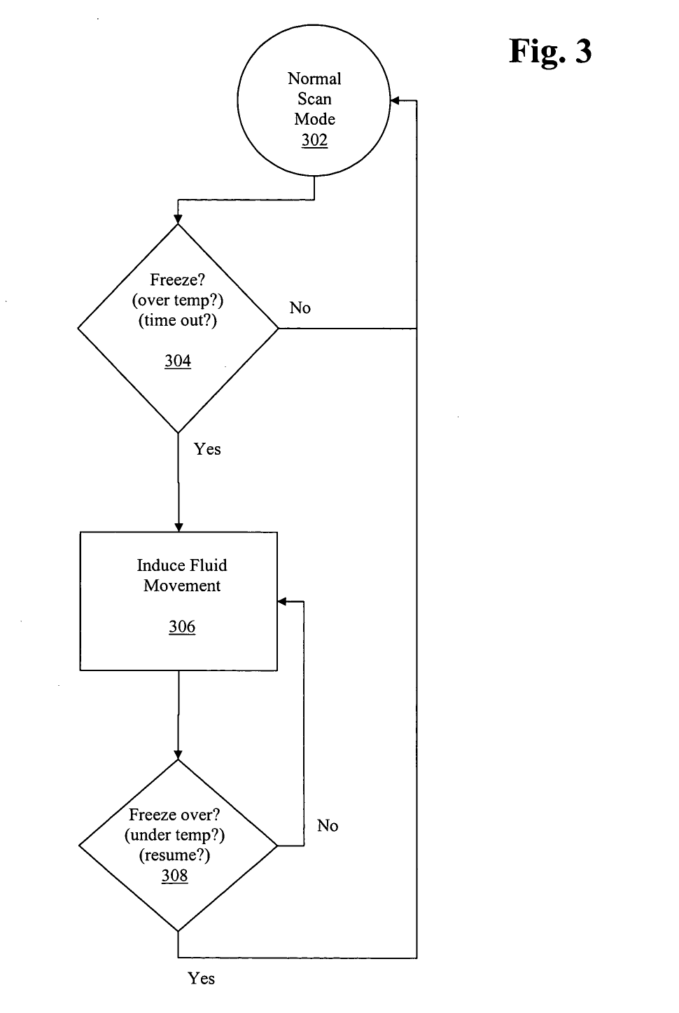

FIG. 3 depicts a flow chart showing exemplary operation of a transducer according to one embodiment. During normal operation of the transducer (block 302), the cooling mechanism is inoperative so as not to interfere with the transducer operation. The system then determines whether the operator has put the transducer in freeze mode, or the system has forced a freeze mode as described above (block 304). In an alternate embodiment, the system may check to see if the transducer temperature has deviated from a pre-defined threshold or if a timer has expired, indicating that cooling of the transducer may be necessary. If the transducer is not in freeze mode, the temperature is within defined limits and/or the timer has not expired, normal operation of the transducer continues. If the transducer is in freeze mode, the transducer temperature has deviated from a defined threshold or a timer has expired, fluid movement mechanism is activated, as described above (block 306). In one embodiment, normal operation of the transducer is inhibited. In an alternate embodiment, where operation of the fluid movement mechanism does not interfere with transducer operation, normal operation of the transducer continues. In one embodiment where the fluid movement mechanism is the transducer itself, any required initialization of transducer movement (for the purposes of imaging) is bypassed and/or deferred so as to immediately begin actuation of the transducer, as described above. If the freeze period ends, the transducer temperature returns within the prescribed limits and/or the timer is reset, the fluid movement mechanism is deactivated and normal transducer operation resumes (if inhibited) (block 308). It will be appreciated that, where the fluid movement mechanism does not interfere with normal transducer operation, the mechanism may continuously operate.

Referring back to FIG. 2, in an alternate embodiment, the cooling mechanism further features a feedback mechanism 112 coupled with one or more thermal sensors 110, such as thermistors, located inside, or in proximity to, the housing 100. The sensor(s) 110 are located so as not to interfere with acoustic operation of the transducer module 11. Via the sensor(s) 110, the feedback mechanism 112 senses the overall temperature, temperature uniformity and/or temperature gradients within the fluid 108, or the lack thereof, to control operation of the fluid movement inducing mechanism 106 and/or actuating mechanism 104 to achieve the desired thermal effect. For example, where the sensor(s) 110 sense that the temperature of the fluid 108 is uniform or has reached an equilibrium, the feedback mechanism 112 may slow or stop fluid movement. Sensing of a dis-equilibrium may cause more aggressive fluid movement. Such a mechanism 112 may be integrated into the transducer housing 100, such as by being mounted externally, or as described below may be integrated with an externally attached cooling mechanism to which the transducer housing 100 is coupled.

In an alternate embodiment, the cooling mechanism described above may be implemented so as to retrofit existing transducers which may or may not already have their own cooling mechanisms. In this embodiment, the cooling mechanism may include a housing or jacket which receives the transducer, i.e., the transducer's housing inserts into the housing/jacket of the cooling mechanism, which also contains the fluid and fluid movement mechanism, thereby effecting a partial or entire immersion of the transducer. The cooling mechanism housing may provide for sealing against the transducer housing to prevent fluid leakage. Once the transducer housing is inserted or contained within the cooling mechanism housing, the transducer is cooled as described above, without substantially interfering with the transducer operation. Control of the cooling mechanism may be integrated into the cooling mechanism housing such as by the integration of thermal sensors which sense the transducer housing temperature and control operation of the fluid movement mechanism in response thereto, as was described above.

It is therefore intended that the foregoing detailed description be regarded as illustrative rather than limiting, and that it be understood that it is the following claims, including all equivalents, that are intended to define the spirit and scope of this invention.

1. A method of cooling an ultrasound transducer:
immersing, at least partially, said transducer in a volume of fluid contained within a housing, said fluid comprising a first portion occupying a first location in said housing, said first location being proximate to said transducer;
receiving thermal energy from said transducer by said first portion;
inducing movement of said fluid within said housing;
moving, in response to said inducing, said first portion having said thermal energy from said first location to at least a second location in said housing, said second location being different from said first location;
moving, in response to said inducing, a second portion of said fluid to said first location; and
receiving thermal energy from said transducer by said second portion.

2. The method of claim 1, wherein said transducer is operative to move within said housing, said inducing further comprising moving said transducer.

3. The method of claim 2, wherein said transducer requires initialization prior to movement, said inducing further comprising bypassing said initialization.

4. The method of claim 1, further comprising:
providing a fluid moving mechanism located in said housing; and
wherein said inducing further comprises activating said fluid moving mechanism.

5. The method of claim 1, further comprising:
sensing a temperature of at least a third portion of said fluid; and
wherein said inducing movement further comprises inducing movement of said fluid based on said sensed temperature.

6. The method of claim 1, wherein at least a third portion said fluid comprises a first phase and a second phase, said inducing further comprising causing said third portion to change from said first phase to said second phase.

7. The method of claim 6, wherein said first phase comprises one of a liquid and solid and said second phase comprises one of liquid and gas.

8. The method of claim 1, wherein said fluid comprises a mixture of liquid and gas.

9. The method of claim 1, further comprising:
operating said transducer to image a subject; and
wherein said inducing further comprises waiting for said operating to cease before inducing said movement.

10. The method of claim 9, wherein said inducing further comprises forcing said operating to cease.

11. An ultrasound transducer comprising:
a housing;
a fluid contained within said housing;
a transducer located in said housing and at least partially immersed in said fluid, wherein said fluid comprises a first portion occupying a first location in said housing, said first location being proximate to said transducer, said first portion operative to receive thermal energy from said transducer; and
a fluid moving mechanism located in said housing and operative to induce movement of said fluid within said housing wherein said first portion having said thermal energy is induced to move from said first location to a second location in said housing, said second location being different from said first location, and further wherein a second portion of said fluid is induced to move to said first location, said second portion being operative to receive thermal energy from said transducer.

12. The ultrasound transducer of claim 11, wherein said transducer is movable within said housing, said fluid moving mechanism comprising said movable transducer.

13. The ultrasound transducer of claim 11, wherein said fluid moving mechanism comprises at least one of a pump, paddle, rotor, impeller, and electrokinetic device.

14. The ultrasound transducer of claim 11, wherein said fluid moving mechanism further comprises a controller and a temperature sensor coupled with said controller and thermally coupled with said fluid to sense a temperature of at least a third portion of said fluid, said controller operative to control said fluid moving mechanism based at least on said sensed temperature.

15. The ultrasound transducer or claim 11, wherein at least a third portion of said fluid comprises a first phase and a second phase, said fluid moving mechanism being further operative to cause said third portion to change from said first phase to said second phase.

16. The ultrasound transducer of claim 15, wherein said first phase comprises one of a liquid and solid and said second phase comprises one of liquid and gas.

17. The ultrasound transducer of claim 11, wherein said fluid comprises a mixture of liquid and gas.

18. An ultrasound transducer comprising:
means for immersing, at least partially, said transducer in a volume of fluid contained within a housing, said fluid comprising a first portion occupying a first location in said housing, said first location being proximate to said transducer such that said first portion receives thermal energy from said transducer;
means for inducing movement of said fluid within said housing, causing said first portion having said thermal energy to move from said first location to at least a second location in said housing, said second location being different from said first location, and causing a second portion of said fluid to move to said first location to receive thermal energy from said transducer.
